(19) United States
(12) Patent Application Publication
(10) Pub. No.: US 2020/0124405 A1
(43) Pub. Date: Apr. 23, 2020

(54) FIBER SENSOR, CURVATURE INFORMATION DERIVATION APPARATUS, ENDOSCOPE SYSTEM, AND METHOD FOR MANUFACTURING FIBER SENSOR

(71) Applicant: OLYMPUS CORPORATION, Tokyo (JP)

(72) Inventors: Ken SATO, Hachioji-shi (JP); Koichi TAKAYAMA, Kokubunji-shi (JP)

(73) Assignee: OLYMPUS CORPORATION, Tokyo (JP)

(21) Appl. No.: 16/724,571

(22) Filed: Dec. 23, 2019

Related U.S. Application Data
(63) Continuation of application No. PCT/JP2017/023391, filed on Jun. 26, 2017.

(51) Int. Cl. G01B 11/24

(52) U.S. Cl. CPC G01B 11/24; A61B 1/00057

(57) ABSTRACT

A fiber sensor includes a light guide provided with a plurality of detection portions in a longitudinal direction of the light guide, and a plurality of detection targets each having a light absorber that absorbs light of a specific wavelength, each of the plurality of detection portions including at least one detection target. Each of the light absorbers contains a color material that reduces an influence of an interaction caused by each detection target at another detection portion existing in the light guide.

This is a Continuation Application of PCT Application No. PCT/JP2017/023391, filed Jun. 26, 2017, the entire contents of which are incorporated herein by reference.

### BACKGROUND

The present invention relates to a fiber sensor in which transmitted light is modulated in accordance with the size of bending, a curvature information derivation apparatus which includes the fiber sensor and derives curvature information including the direction of bending and the size of bending by detecting the transmitted light, an endoscope system including the curvature information derivation apparatus, and a method for manufacturing the fiber sensor.

There is known a curvature information derivation apparatus which is incorporated in an insertion device having a flexible insertion portion, e.g., an insertion portion of an endoscope, and detects the curvature information. For example, Japanese Patent No. 4714570 discloses an endoscope shape detection probe as a fiber sensor used in a curvature information derivation apparatus. This probe has an optical fiber which is incorporated in an insertion portion of an endoscope and is bent integrally with the insertion portion. The optical fiber is provided with two light modulators for detecting curvatures in two directions substantially orthogonal to each other at substantially the same position in the longitudinal direction of the optical fiber. The light modulator modulates the intensity of a wavelength component of the light transmitted through the optical fiber. The curvature information derivation apparatus derives, as curvature information, the curvature of the optical fiber in the light modulator, and hence the curvature of the insertion portion that is bent integrally with the optical fiber, based on a change in the intensity of wavelength components before and after passing through the light modulator of this probe.

Furthermore, in the curvature information derivation apparatus, it is possible to derive curvature information at a plurality of portions by arranging a plurality of light modulators that optically modulate wavelength components different from each other at different positions in the longitudinal direction of the optical fiber.

The endoscope shape detection probe disclosed in Japanese Patent No. 4714570 is a transmission system in which light is transmitted through an optical fiber in one direction. In contrast, a curvature information derivation apparatus using a reflective fiber sensor as disclosed in WO 2016/178279 A1 is also known which reflects light transmitted through an optical fiber by a reflection member provided at the distal end of the optical fiber and returns the reflected light to the proximal end side of the optical fiber. In a reflective fiber sensor as in WO 2016/178279 A1, since light transmitted through an optical fiber is subjected to light modulation twice, the bending state is reflected more strongly than in transmission systems, so that curvature information can be derived more easily and more accurately than in transmission systems.

### SUMMARY

According to an exemplary embodiment, a fiber sensor includes a light guide provided with a plurality of detection portions in a longitudinal direction of the light guide, and a plurality of detection targets each having a light absorber that absorbs light of a specific wavelength, each of the plurality of detection portions including at least one detection target, wherein each of the light absorbers contains a color material that reduces an influence of an interaction caused by each detection target at another detection portion existing in the light guide.

According to an exemplary embodiment, an endoscope system includes a curvature information derivation apparatus that includes the fiber sensor according to the exemplary embodiment, and derives curvature information including a direction of bending and a size of bending by detecting light transmitted by the light guide of the fiber sensor; and an endoscope including an insertion portion in which the light guide is incorporated.

According to an exemplary embodiment, a curvature information derivation apparatus includes a fiber sensor including a light guide provided with a plurality of detection portions in a longitudinal direction of the light guide, and a curvature information calculator configured to derive curvature information of each of the plurality of detection portions, wherein each of the plurality of detection portions including at least one detection target having a light absorber that absorbs light of a specific wavelength, each of the light absorbers is affected by an interaction caused by a detection target at another detection portion, and the curvature information calculator derives the curvature information of each of the plurality of detection portions by expressing a curved component with an equation including curvature information of all of detection portions existing in the light guide.

According to an exemplary embodiment, an endoscope system includes the curvature information derivation apparatus according to the exemplary embodiment, and an endoscope including an insertion portion in which the light guide is incorporated.

According to an exemplary embodiment, a method for manufacturing a fiber sensor including a light guide which has a core, a clad surrounding the core and a jacket surrounding the clad, and is provided with a plurality of detection portions in a longitudinal direction of the light guide includes removing the jacket and the clad corresponding to each of a plurality of detection targets to expose the core, each of the plurality of detection portions including at least one detection target, and providing a light absorber on the core corresponding to each of the detection targets and exposed, wherein each of the light absorbers contains a color material that reduces an influence of an interaction caused by each detection target at another detection portion existing in the light guide.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram schematically showing an example of an endoscope system including a curvature information derivation apparatus according to a first embodiment of the present invention.

FIG. 2 is a block diagram illustrating an example of a fiber sensor of the curvature information derivation apparatus.

FIG. 3A is a cross-sectional view including an optical axis of a light guide of a sensor unit.

FIG. 3B is a cross-sectional view of the light guide in a radial direction taken along line B-B in FIG. 3A.

FIG. 4 is a diagram for explaining two detection portions each having two detection targets in a scope where the light guide can be regarded as being in the same bending state when it is curved.

FIG. 5 is a diagram for explaining two detection portions each having one detection target in a scope where the light guide can be regarded as being in the same bending state when it is curved.

FIG. 6A is a diagram illustrating an example of an arrangement relationship of detection targets when two detection targets are included in one detection portion.

FIG. 6B is a diagram illustrating another example of the arrangement relationship of detection targets when two detection targets are included in one detection portion.

FIG. 6C is a diagram illustrating another example of the arrangement relationship of detection targets when two detection targets are included in one detection portion.

FIG. 7A is a diagram for explaining a case where the correlation between the light absorption property in a transmission system and the light absorption property in a reflection system is strong.

FIG. 7B is a diagram for explaining a case where the correlation between the light absorption property in a transmission system and the light absorption property in a reflection system is weak.

FIG. 8 is a diagram illustrating light absorption properties of four light absorbers that can be used for the four detection targets in FIG. 4.

FIG. 9 is a diagram showing a table of combinations of arrangements of the four light absorbers of FIG. 8.

FIG. 10 is a diagram showing a table of correlation coefficients in the combinations shown in FIG. 9.

FIG. 11 is a diagram for explaining three detection portions each having two detection targets in a scope where the light guide can be regarded as being in the same bending state when it is curved.

FIG. 12A is a diagram for explaining an example of an arrangement relationship of the four detection targets shown in FIG. 4 in a cross section of the light guide in the radial direction.

FIG. 12B is a diagram for explaining another example of the arrangement relationship of the four detection targets shown in FIG. 4 in a cross section of the light guide in the radial direction.

FIG. 13 is a diagram showing a table of combinations of the arrangements of respective detection targets in the combination 2 shown in FIG. 9.

FIG. 14 is a diagram showing a table summarizing the combinations of the arrangements of the detection targets shown in FIG. 13.

FIG. 15 is a diagram showing a table of correlation coefficients in the combinations shown in FIG. 14.

FIG. 16 is a diagram for explaining a combination of light absorbers in the case of three detection portions each having two detection targets.

FIG. 17 is a diagram for explaining an example of an arrangement relationship of six detection targets shown in FIG. 16 in a radial cross section of the light guide.

### DETAILED DESCRIPTION

### First Embodiment

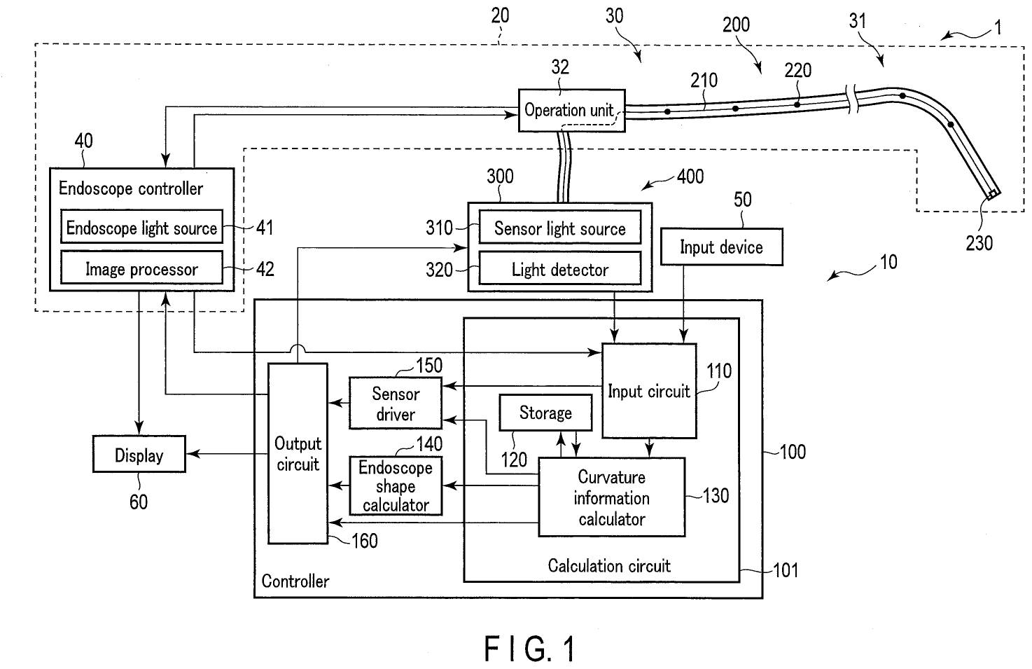

FIG. 1 is a diagram schematically showing an example of an endoscope system 1 including a curvature information derivation device 10 according to a first embodiment of the present invention. An endoscope system 1 includes a curvature information derivation apparatus 10, an endoscope apparatus 20, an input device 50, and a display 60. The endoscope apparatus 20 includes an endoscope 30 and an endoscope controller 40. The endoscope 30 is connected to the endoscope controller 40 via a universal cord (not shown).

The endoscope 30 includes an insertion portion 31 to be inserted into an insertion object, and an operation unit 32 connected to the proximal end side of the insertion portion 31. The insertion portion 31 is an elongated tubular portion on the distal end side of the endoscope and has flexibility. The insertion portion 31 incorporates an illumination optical system, an observation optical system, an imaging element, etc. (not shown) at the distal end. The insertion portion 31 includes a bending portion that bends in a desired direction when the user operates the operation unit 32. Various operations of the endoscope 30 including the bending operation are input to the operation unit 32.

The endoscope controller 40 includes an endoscope light source 41 for supplying illumination light to the illumination optical system of the endoscope 30. The endoscope light source 41 includes general light emitting elements, such as a halogen lamp, a xenon lamp, a laser diode (LD), and a light emitting diode (LED). The endoscope controller 40 controls various operations of the endoscope 30 and the endoscope light source 41, such as drive control of an imaging element in the endoscope 30, drive control of the endoscope light source 41, and control of light modulation of illumination light from the endoscope light source 41. In addition, the endoscope controller 40 includes an image processor 42 for processing an image acquired by an observation optical system and the imaging element of the endoscope 30.

The curvature information derivation apparatus 10 is an apparatus for deriving curvature information of the insertion portion 31 of the endoscope 30. In this specification, a direction of bending and a size of bending are collectively referred to as curvature information. The curvature information derivation apparatus 10 includes a controller 100 and a fiber sensor 400 including a sensor unit 200 and a sensor controller 300. Details of these members will be described later.

The input device 50 is a general input equipment, such as a keyboard and a mouse. The input device 50 is connected to the controller 100 of the curvature information derivation apparatus 10. The input device 50 is used for a user to input various commands for operating the curvature information derivation apparatus 10. The input device 50 may be a storage medium. In this case, information stored in the storage medium is input in the controller 100.

The display 60 is a general monitor, such as a liquid crystal display. The display 60 is connected to the endoscope controller 40 and displays an observation image acquired by the endoscope 30. The display 60 is connected to the curvature information derivation apparatus 10 and displays curvature information obtained from the curvature information derivation apparatus 10, a curved shape of the insertion portion 31, etc.

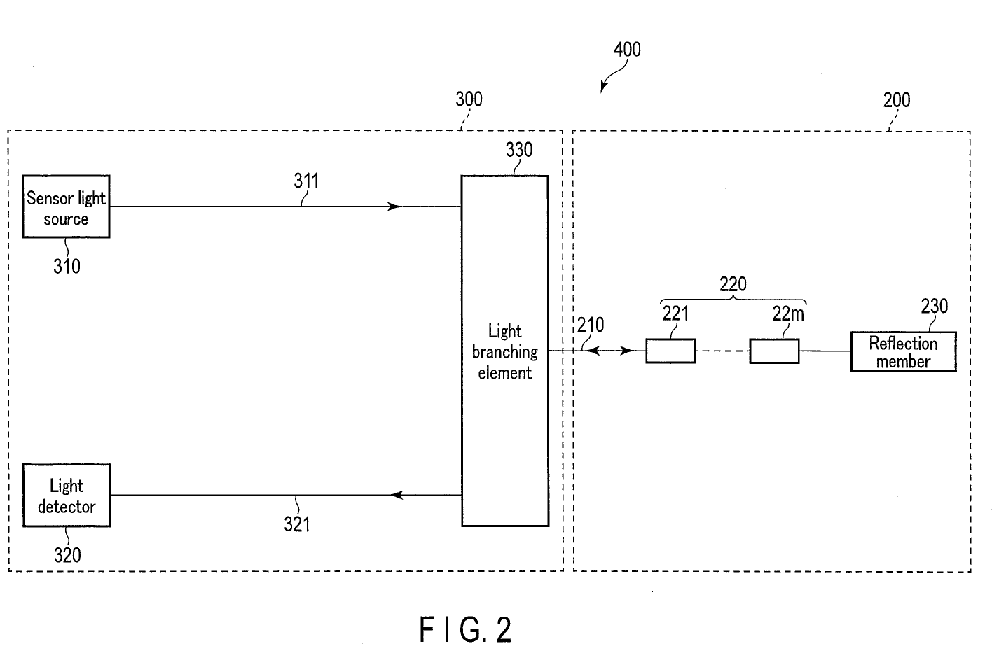

Next, a fiber sensor 400 of the curvature information derivation apparatus 10 will be described. FIG. 2 is a block diagram illustrating an example of a fiber sensor 400 including the sensor unit 200 and the sensor controller 300. The sensor unit 200 includes a light guide 210, a plurality of detection targets 220 provided in the light guide 210, and a reflection member 230. The sensor controller 300 includes a sensor light source 310, a light detector 320, and a light branching element 330.

The light guide 210 is an optical fiber, for example, and has flexibility. The proximal end of the light guide 210 is connected to the light branching element 330 of the sensor controller 300. As schematically shown in FIG. 1, the light guide 210 is incorporated in the insertion portion 31 of the endoscope 30 along the longitudinal direction thereof. The plurality of detection targets 220 of the light guide 210 are arranged at points or across a region of the insertion portion 31 where curvature information should be obtained.

FIGS. 1 and 2 show a plurality of detection targets 220. These detection targets 220 include a first detection target 221 and can further include an mth detection target 22m. That is, m detection portions 220 can be provided on the light guide 210. Here, m is an arbitrary number. For example, the m detection targets 221 to 22m are arranged at different positions in the longitudinal direction (optical axis direction) of the light guide 210, that is, arranged to be spaced apart from each other.

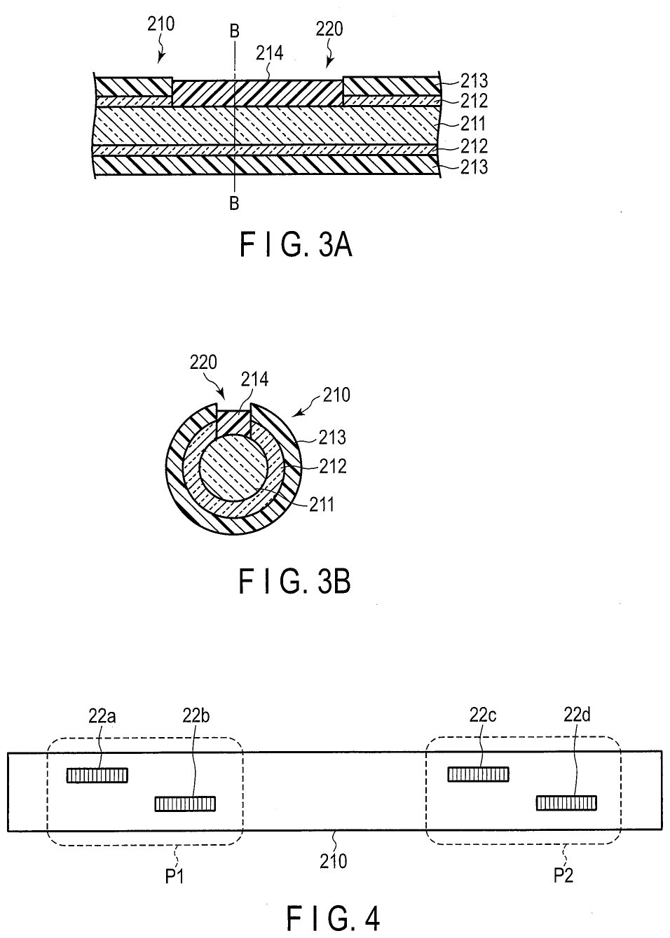

FIG. 3A is a cross-sectional view including an optical axis of the light guide 210. FIG. 3B is a cross-sectional view of the light guide 210 in the radial direction taken along a B-B line in FIG. 3A. The light guide 210 has a three-layer structure including a core 211, a clad 212 surrounding the core 211, and a jacket (covering/buffer) 213 surrounding the clad 212.

Each detection target 220 modulates light guided in the light guide 210 according to the bending state. For example, when the light quantity (light intensity) is modulated, a light absorber 214 is disposed in the detection target 220. That is, the detection target 220 is formed by removing a part of the jacket 213 and a part of the clad 212 to expose the core 211 and providing the light absorber 214 on the exposed core 211. For a light absorbing agent constituting the light absorber 214, a substance colored with a color material (pigment) whose refractive index is larger than the refractive index of the core 211 and smaller than the refractive index of the jacket 213 is used. For the color material, for example, dyes, pigments, and metal nanoparticles are used.

The reflection member 230 is connected to the distal end of the light guide 210, i.e., on the side not connected to the light branching element 330 in the sensor controller 300. The reflection member 230 is a mirror in which a reflective material, such as silver, is applied to the reflecting surface. The reflection member 230 reflects light transmitted from the proximal end of the light guide 210 to the distal end so as to return it in a direction where the light branching portion 330 is present. That is, the reflection member 230 reflects the light entering in the light guide 210 and returns it to the entrance side. In order to prevent oxidation or sulfuration of the reflective material, an inhibitor may be mixed in the reflective material. Alternatively, a coating agent may be applied to the surface of the reflective material in order to prevent oxidation or sulfuration of the reflective material.

Examples of the sensor light source 310 (hereinafter simply referred to as a light source 310) include, for example, a general light emitting element, such as a halogen lamp, a xenon lamp, a laser diode (LD), and a light emitting diode (LED). The light intensity of light emitted from the light source 310 is preferably uniform in a wavelength band detected by the light detector 320. The light intensity here is not limited to the absolute intensity. It may be a state where the output of the light detector 320 for each wavelength may be uniform according to the spectral sensitivity of the light detector 320. Furthermore, a filter may be inserted into an optical path between the light source 310 and the light detector 320 so that the spectrum of light emitted from the light source 310 and entered in the light detector 320 is uniform.

The light detector 320 is a detector that acquires a spectrum (the relationship between a light quantity (light intensity) and a wavelength) of light that has passed through the detection targets 220 and has been returned by the reflection member 230. Specifically, the light detector 320 can be configured by a spectroscope that acquires light intensity for each wavelength (wavelength band). A combination of color filters and light receiving elements may be used as long as the light intensity for each wavelength band can be acquired.

The light branching element 330 is a branching unit that branches light transmitted from the light source 310 to the sensor unit 200 via a light guide 311 and light transmitted from the sensor unit 200 to the light detector 320 via a light guide 321, and includes optical couplers, half mirrors, etc. The light guides 311 and 321 may also be optical fibers having flexibility.

The operation of the fiber sensor 400 will be described. The light source 310 emits light in a predetermined light-emitting wavelength range. The emitted light is guided from the light guide 311 to the light guide 210 via the light branching element 330, reflected by the reflection member 230 and turned back, again guided from the light guide 210 to the light guide 321 via the light branching element 330, and reaches the light detector 320. The light detector 320 detects light quantity information indicating a spectrum of light that has passed through the detection targets 220 (221 to 22m) and has been reflected by the refection member 230 and returned, i.e., a relationship between a wavelength and a light intensity (light quantity), in a predetermined wavelength range.

Here, a change in the amount of light absorbed by the light absorber 214 of each detection target 220 due to a change in the bending state of each detection target 220 will be described. The light absorber 214 absorbs light having a predetermined wavelength (wavelength band) among the light transmitted through the light guide 210. For example, when the detection target 220 is in a linear state, part of the light guided through the light guide 210 is absorbed by the light absorber 214. Meanwhile, when the light guide 210 is curved so that the detection target 220 is located inside a bend, the quantity of light hitting against the light absorber 214 decreases, so the quantity of light absorbed by the light absorber 214 becomes small. Therefore, the amount of light transmitted through the light guide 210 becomes larger than that in the linear state. In contrast, when the light guide 210 is curved so that the detection target 220 is located outside the bend, the quantity of light hitting against the light absorber 214 increases, so the amount of light absorbed by the light absorber 214 increases. Therefore, the amount of light transmitted through the light guide 210 becomes smaller than that in the linear state.

As described above, the detection target 220 modulates light transmitted through the light guide 210 according to the bending state of the detection target 220. In the present embodiment, the light absorber 214 of the detection target 220 modulates the amount of light (light intensity) transmitted through the light guide 210. In other words, the amount of light transmitted through the light guide 210 changes because the amount of light absorbed by the light absorber 214 of the detection target 220 changes according to the bending state of the detection target 220. The curvature information derivation device 10 derives curvature information of the respective detection targets 220 by using this light amount change, i.e., based on a spectrum detected by the light detector 320, in short, the detected light quantity information.

In the case where the light guide 210 is provided with a plurality of detection targets 221 to 22m, for example, the light absorbers 214 having different light absorption rates at respective wavelengths, that is, having different light modulation properties, are applied to the respective detection targets 220. That is, the same number of different types of light absorbers 214 as the number of detection targets 221 to 22m can be prepared. In this case, each light absorber 214 is colored with a different color material so that the properties of absorption spectra (the relationship between a wavelength and a light absorption amount) of respective light absorbers 214 are different in each of the detection targets 221 to 22m.

Here, in order to prevent oxidation or sulfuration of the color material, the following methods can be taken. One method is to mix an inhibitor in a color material. Another method is to apply a coating agent to the surfaces of the light absorbers 214. Another method is to cover a part of the sensor unit 200 including the detection targets 220 or the sensor unit 200 with a coating. Another method is to enclose an inhibitor in a coating covering in a part of the sensor unit 200 including the detection targets 220 or the sensor unit 200. Another method is to enclose nitrogen gas in a coating covering in a part of the sensor unit 200 including the detection targets 220 or the sensor unit 200.

Furthermore, when the sensor unit 200 is detachable from the insertion portion 31 (attached to a channel or the like), in order to prevent deterioration due to light, oxidation, sulfuration, etc., of the sensor unit 200, the sensor unit 200 is preferably wrapped (packaged) using an aluminum film, etc., and stored during the storage of the sensor unit 200. Furthermore, an antioxidant, a dehumidifier, nitrogen gas, or the like may be enclosed in the storage package. The place at which the sensor unit 200 is detachable from the insertion portion 31 is not limited to the place between the optical branching element 330 and the sensor part 200. The place may be between the light branching element 330 and the sensor light source 310 or between the light branching element 330 and the light detector 320.

Next, the controller 100 of the curvature information derivation apparatus 10 will be described with reference to FIG. 1 again. The controller 100 is configured by an electronic computer which is a personal computer, for example. The controller 100 includes an input circuit 110, a storage 120, a curvature information calculator 130, an endoscope shape calculator 140, a sensor driver 150, and an output circuit 160. Among these, the input circuit 110, the storage 120, and the curvature information calculator 130 constitute a calculation circuit 101. The controller 100 is communicatively connected to the endoscope controller 40. In FIG. 1, the controller 100 of the curvature information derivation apparatus 10 and the endoscope controller 40 are separated, but the controller 100 may be incorporated in the endoscope controller 40.

The above-described detected light quantity information is input in the input circuit 110 from the light detector 320 of the sensor controller 300. The input circuit 110 transmits the detected light quantity information to the curvature information calculator 130. The information output from the endoscope controller 40 is also input in the input circuit 110. Alternatively, information input in the input device 50 is also input in the input circuit 110. The input circuit 110 transmits a signal including the input information to the curvature information calculator 130 or the sensor driver 150.

The storage 120 stores various information necessary for calculations performed by the curvature information calculator 130. The storage 120 stores, for example, programs including calculation algorithms.

The curvature information calculator 130 calculates curvature information of each detection target 220 based on information, such as detected light quantity information acquired via the input circuit 110, and information, calculation formulas, etc. stored in the storage 120. The curvature information calculator 130 transmits the calculated curvature information of the respective detection targets 220 to the endoscope shape calculator 140 and the output circuit 160. In addition, the curvature information calculator 130 outputs, to the sensor driver 150, information related to the operation of the light detector 320 necessary for calculating curvature information, such as the gain of the light detector 320.

The endoscope shape calculator 140 includes, for example, a CPU or an ASIC. The endoscope shape calculator 140 calculates the shape of the insertion portion 31 of the endoscope 30 in which the detection targets 220 are arranged, based on the curvature information of each detection target 220 calculated by the curvature information calculator 130. The calculated shape of the insertion portion 31 is transmitted to the output circuit 160. The endoscope shape calculator 140 may be incorporated in the curvature information calculator 130.

The sensor driver 150 generates a drive signal for the light detector 320 based on information acquired from the input circuit 110 or the curvature information calculator 130. By this drive signal, the sensor driver 150 switches the on/off of the light detector 320 based on, for example, a user instruction input to the input device 50 or adjusts the gain of the light detector 320 based on the information acquired from the curvature information calculator 130. The sensor driver 150 also controls the operation of the light source 310. The sensor driver 150 transmits the generated drive signal to the output circuit 160.

The output circuit 160 outputs, to the display 60, the curvature information of the detection targets 220 acquired from the curvature information calculation circuit 130 or the shape of the insertion portion 31 acquired from the endoscope shape calculator 140. Furthermore, the output circuit 160 outputs, to the endoscope controller 40, the curvature information of the detection targets 220 acquired from the curvature information calculation circuit 130 or the shape of the insertion portion 31 acquired from the endoscope shape calculator 140. Furthermore, the output circuit 160 outputs a drive signal from the sensor driver 150 to the light detector 320.

Next, operations of the endoscope system 1 and the curvature information derivation apparatus 10, according to the present embodiment, will be described.

The insertion portion 31 of the endoscope 30 is inserted into an insertion body by the user. At this time, the insertion portion 31 bends following the bending state of the insertion body. The endoscope 30 obtains an image signal by an observation optical system and an imaging element provided at the distal end of the insertion portion 31, and the obtained image signal is transmitted to the endoscope controller 40. The endoscope controller 40 creates an observation image by means of the image processor 42 based on the acquired image signal, and causes the display 60 to display the created observation image.

When the user wants to display curvature information of the insertion portion 31 of the endoscope 30 on the display 60 or when the user wants the endoscope controller 40 to perform various operations using the curvature information of the insertion portion 31, the user inputs this to the controller 100 through the input device 50. At this time, the curvature information derivation apparatus 10 operates.

When the curvature information derivation apparatus 10 operates, the light source 310 of the sensor controller 300 is activated based on a drive signal transmitted to the sensor controller 300 from the sensor driver 150 via the output circuit 160. The light source 310 emits light in a predetermined light-emitting wavelength range. Then, as described above, the amount of light transmitted through the light guide 210 changes according to the bending state of each detection target 220, and the changed light intensity is detected for each wavelength by the light detector 320. That is, the light detector 320 acquires detected light quantity information.

The light detector 320 transmits the acquired detected light quantity information to the input circuit 110 of the controller 100. The transmitted detected light quantity information is acquired by the curvature information calculator 130, and the curvature information calculator 130 calculates curvature information (the direction of bending and the size of bending) of each detection target 220. A specific calculation method of the curvature information at the curvature information calculator 130 will be described later.

The curvature information of each detection target 220 calculated by the curvature information calculator 130 is acquired by the endoscope shape calculator 140. The endoscope shape calculator 140 calculates the shape of the insertion portion 31 of the endoscope 30 based on the curvature information of the respective detection targets 220.

The curvature information of each detection target 220 calculated by the curvature information calculator 130 or the shape of the insertion portion 31 calculated by the endoscope shape calculator 140 is acquired by the endoscope controller 40 via the output circuit 160. The endoscope controller 40 controls the operation of the endoscope 30 based on the curvature information of the detection target 220 or the shape of the insertion portion 31.

Also, the curvature information of each detection target 220 calculated by the curvature information calculator 130 or the shape of the insertion portion 31 calculated by the endoscope shape calculator 140 is displayed on the display 60 via the output circuit 160.

Furthermore, the information input in the input circuit 110 and the curvature information of each detection target 220 calculated by the curvature information calculator 130 are acquired by the sensor driver 150. Based on the acquired information, the sensor driver 150 transmits a drive signal to the light detector 320 via the output circuit 160 to control the operation of the light detector 320.

As described above, in the curvature information derivation apparatus 10, the curvature information of each detection target 220 is derived by the curvature information calculator 130. The endoscope shape calculator 140 calculates the shape of the insertion portion 31 of the endoscope 30 based on the derived curvature information of each detection target 220. Thereby, the user can obtain the curvature information of each detection target 220 or the shape of the insertion portion 31 during the operation of the endoscope 30. Furthermore, the endoscope controller 40 can appropriately control the operation of the endoscope 30 according to the calculated curvature information of each detection target 220 or the shape of the insertion portion 31.

The light absorption at the curved portion changes under the influence of the shape of another curved portion (light absorption at another curved portion). That is, the absorption of light at a certain curved portion interacts with the absorption of light at another curved portion. Due to this interaction, the detected light quantity information acquired by the light detector 320 may not accurately indicate the bending state of each detection target 220. When the curvature information calculator 130 calculates curvature information based on such detected light quantity information, an error occurs in the obtained curvature information.

Therefore, the present embodiment is configured such that even when there is an interaction, the curvature information can be correctly derived (an error is minimized) by selecting a color material of the light absorber 214 which is less affected by the interaction. This will be described in detail below.

Here, as shown in FIG. 4, it is assumed that there are two detection portions P1 and P2 in the light guide 210. A detection portion is a curved portion (section or region) that is considered to have the same direction of bending and the same size in each region.

In the example of FIG. 4, the detection portion P1 includes detection targets 22a and 22b, and the detection targets constitute a detection target group ab. The detection portion P2 includes detection targets 22c and 22d, and the detection targets constitute a detection target group cd. The light absorbers 214 of the detection targets 22a, 22b, 22c and 22d are colored with different color materials.

In a transmissive fiber sensor in which light is transmitted in one direction through the light guide 210, a change in the light quantity resulting from the curvature of the detection target group ab at the detection portion P1 occurs based on the light absorption properties of the respective light absorbers 214 arranged in the detection target 22a and the detection target 22b. This is because, as the light absorbers 214 of the respective detection targets 22a, 22b, 22c and 22d, those having light absorption properties in consideration of the absorption wavelengths of the other light absorbers 214 are selected.

However, it has been found that the light absorption property differs between a transmission system and a reflection system. Therefore, in the reflection-type fiber sensor 400 in which light is transmitted in the opposite direction through the light guide 210 using the reflection member 230 as in the present embodiment, the change in light quantity resulting from the curvature of the detection target group ab at the detection portion P1 is affected by the bending state of the detection target group cd at the detection portion P2. That is, a change in light quantity occurs based not only on the change in light quantity resulting from the curvature of the detection target group ab at the detection portion P1 but also the light absorption property of the detection targets 22c and 22d at the detection portion P2.

Similarly, the change in light quantity due to the curvature of the detection target group cd at the detection portion P2 is also affected by the bending state of the detection target group ab at the detection portion P1.

As described above, in the reflection type fiber sensor 400, a change in the light quantity has an interaction.

This interaction is greatly influenced by the scattering of the light absorber 214 of the respective detection targets 220. As described above, as the color material contained in the light absorber 214, for example, a dye, a pigment, and metal nanoparticles are used. When the particle size of these color materials is large, scattering tends to occur.

Therefore, the size of the particles of these color materials is made smaller than the wavelength included in the wavelength band used for detection, i.e., the wavelength of light detected by the light detector 320. Thereby, scattering can be reduced. In particular, it is preferable that the size of the color material particles is ½ or less the wavelength as the effect of scattering can thereby be reduced further.

As described above, by limiting the size of the color material particles, light scattering by the light absorber 214 is reduced, and the influence of the interaction is reduced. As a result, the detected light quantity information acquired by the light detector 320 accurately indicates the bending state of each detection target 220, and the curvature information calculator 130 can determine the curvature information correctly (the error of the determined curvature information can be reduced).

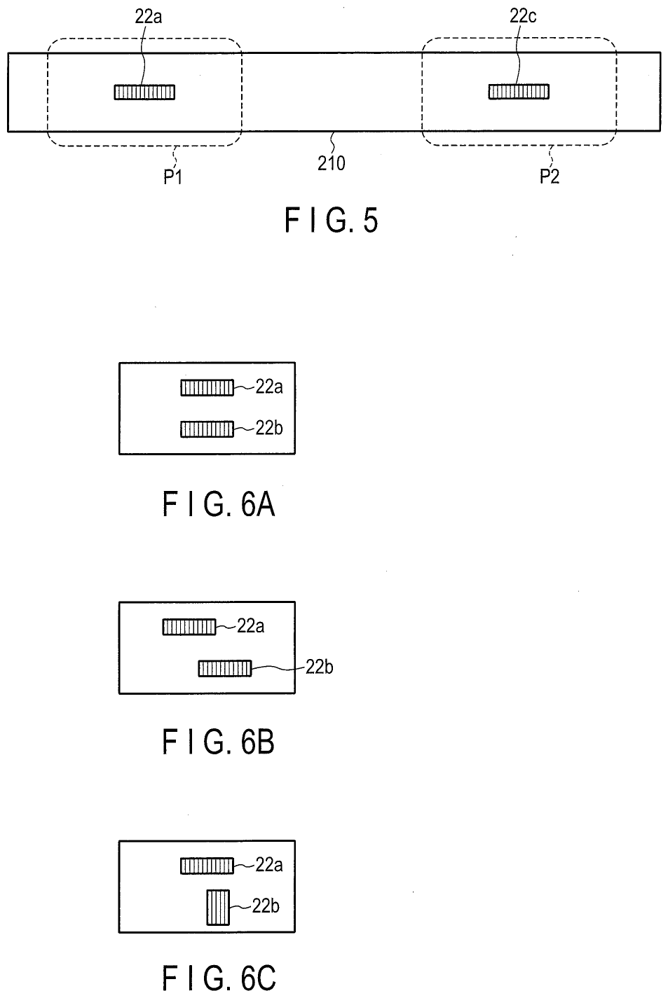

In addition, as shown in FIG. 4, the case where two detection targets (22a and 22b, 22c and 22d) are included in each of the two detection portions P1 and P2 has been described. In the case of only bending within a plane, for example, only bending in the vertical direction as in a pyeloscope, when one detection target (22a, 22c) is included in each of the two detection portions P1, P2, as shown in FIG. 5, it is also preferable to limit the size of each color material particle, similarly. However, in the case where the two detection targets 22a and 22c are partitioned and arranged in the same color, there is no need for this because no interaction occurs.

When two detection targets are included in one detection portion, two detection targets, for example, the detection targets 22a and 22b may be arranged at the same position in the longitudinal direction of the light guide 210, as shown in FIG. 6A. Alternatively, as shown in FIG. 6B, the two detection targets 22a and 22b may be arranged so that the positions thereof in the longitudinal direction of the light guide 210 may overlap. Furthermore, as shown in FIG. 6C, the two detection targets 22a and 22b may be different from each other in the length and/or width in the longitudinal direction of the light guide 210.

In addition, when only one detection portion exists in the light guide 210, the interaction does not occur even if two detection targets of different colors are included therein, so that there is no need to limit the size of the color material particles.

As described above, light scattering by the light absorber 214 is reduced, and the influence of the interaction is reduced. However, it is not possible to completely eliminate the influence of interaction.

The curvature information calculator 130 can determine curvature information correctly by adopting the following calculation method of curvature information (an error is minimized), even when there is such an interaction.

Here, a light quantity change rate at each wavelength (or a logarithm of the light quantity change rate) V, a curved component S indicating the bending state of each detection target 220, and a coefficient R for separating the V into curved components S of respective detection targets 220 are defined as the following equations (1) to (3). The light quantity change rate V can be obtained based on the detected light quantity information acquired by the light detector 320, and the curved component S is a function of the curvature information (a direction of bending κ and a size of bending (curvature) θ).

$$\begin{matrix}V = [ \begin{matrix}{V}_{λ1} \\ {V}_{λ2} \\ ⋮ \\ {V}_{λ n}\end{matrix} ] & ( 1 ) \\ S = [ \begin{matrix}{S}_{a} \\ {S}_{b} \\ ⋮ \\ {S}_{X}\end{matrix} ] & ( 2 ) \\ R = [ \begin{matrix}{R}_{a λ1} & {R}_{a λ 2} & … & {R}_{a λ n} \\ {R}_{b λ2} & ⋱ & & \\ ⋮ & & & \\ {R}_{X λ 1} & & & {R}_{X λ n}\end{matrix} ] & ( 3 )\end{matrix}$$

The curvature information calculator 130 separates the detected light quantity information acquired by the light detector 320 into a curved component S of each detection target 220 by the following equation (4).

S=RV (4)

Hereinafter, as a specific example, a configuration having four detection targets 22a, 22b, 22c, and 22d as shown in FIG. 4 will be described. Two detection targets 220 (two detection portions P1 and P2) are arranged on the light guide 210. A detection target group ab at the detection portion P1 is curved with a curvature θ₁ and a direction of bending κ₁, and a detection target group cd at the detection portion P2 is curved with a curvature θ₂ and a direction of bending κ₂.

In this case, each curved component S can be expressed by a function of the curvature information pertaining thereto and other curvature information as shown in the following equation (5).

$$S = [ \begin{matrix}{S}_{a} ( {κ}_{1} , {θ}_{1} , {κ}_{2} , {θ}_{2} ) \\ {S}_{b} ( {κ}_{1} , {θ}_{1} , {κ}_{2} , {θ}_{2} ) \\ {S}_{c} ( {κ}_{1} , {θ}_{1} , {κ}_{2} , {θ}_{2} ) \\ {S}_{d} ( {κ}_{1} , {θ}_{1} , {κ}_{2} , {θ}_{2} )\end{matrix} ] = [ \begin{matrix}{f}_{a} ( {κ}_{1} , {θ}_{1} ) + {g}_{a} ( {κ}_{2} , {θ}_{2} ) \\ {f}_{b} ( {κ}_{1} , {θ}_{1} ) + {g}_{b} ( {κ}_{2} , {θ}_{2} ) \\ {f}_{c} ( {κ}_{1} , {θ}_{1} ) + {g}_{c} ( {κ}_{2} , {θ}_{2} ) \\ {f}_{d} ( {κ}_{1} , {θ}_{1} ) + {g}_{d} ( {κ}_{2} , {θ}_{2} )\end{matrix} ] \qquad ( 5 )$$

By expressing the curved component S as an equation including the curvature information of all of the detection portions as shown in equation (2), curvature information can be derived even if there is an interaction. For this reason, curvature information can be calculated correctly (an error is minimized).

Although FIG. 4 shows an example of the two detection portions P1 and P2, when there are three detection portions on the light guide 210, it may be expressed similarly as in equation (3). At this time, it is assumed that a detection target group ef constituting the third detection portion is curved with a curvature θ₃ and a direction of bending κ₃. That is, in this case, the curved component S can be expressed as the following equation (6).

$$\begin{matrix}S = [ \begin{matrix}{S}_{a} ( {κ}_{1} , {θ}_{1} , {κ}_{2} , {θ}_{2} , {κ}_{3} , {θ}_{3} ) \\ {S}_{b} ( {κ}_{1} , {θ}_{1} , {κ}_{2} , {θ}_{2} , {κ}_{3} , {θ}_{3} ) \\ {S}_{c} ( {κ}_{1} , {θ}_{1} , {κ}_{2} , {θ}_{2} , {κ}_{3} , {θ}_{3} ) \\ {S}_{d} ( {κ}_{1} , {θ}_{1} , {κ}_{2} , {θ}_{2} , {κ}_{3} , {θ}_{3} ) \\ {S}_{e} ( {κ}_{1} , {θ}_{1} , {κ}_{2} , {θ}_{2} , {κ}_{3} , {θ}_{3} ) \\ {S}_{f} ( {κ}_{1} , {θ}_{1} , {κ}_{2} , {θ}_{2} , {κ}_{3} , {θ}_{3} )\end{matrix} ] \\ = [ \begin{matrix}{f}_{a} ( {κ}_{1} , {θ}_{1} ) + {g}_{a} ( {κ}_{2} , {θ}_{2} ) + {h}_{a} ( {κ}_{3} , {θ}_{3} ) \\ {f}_{b} ( {κ}_{1} , {θ}_{1} ) + {g}_{b} ( {κ}_{2} , {θ}_{2} ) + {h}_{b} ( {κ}_{3} , {θ}_{3} ) \\ {f}_{c} ( {κ}_{1} , {θ}_{1} ) + {g}_{c} ( {κ}_{2} , {θ}_{2} ) + {h}_{c} ( {κ}_{3} , {θ}_{3} ) \\ {f}_{d} ( {κ}_{1} , {θ}_{1} ) + {g}_{d} ( {κ}_{2} , {θ}_{2} ) + {h}_{d} ( {κ}_{3} , {θ}_{3} ) \\ {f}_{e} ( {κ}_{1} , {θ}_{1} ) + {g}_{e} ( {κ}_{2} , {θ}_{2} ) + {h}_{e} ( {κ}_{3} , {θ}_{3} ) \\ {f}_{f} ( {κ}_{1} , {θ}_{1} ) + {g}_{f} ( {κ}_{2} , {θ}_{2} ) + {h}_{f} ( {κ}_{3} , {θ}_{3} )\end{matrix} ]\end{matrix} \qquad ( 6 )$$

The same applies to the case where the light guide 210 has more detection portions.

Furthermore, each curved component S may be expressed as in the following equation (7). Compared with the equation (5), more accurate curvature information can be obtained.

$$\begin{matrix}S = [ \begin{matrix}{S}_{a} ( {κ}_{1} , {θ}_{1} , {κ}_{2} , {θ}_{2} ) \\ {S}_{b} ( {κ}_{1} , {θ}_{1} , {κ}_{2} , {θ}_{2} ) \\ {S}_{c} ( {κ}_{1} , {θ}_{1} , {κ}_{2} , {θ}_{2} ) \\ {S}_{d} ( {κ}_{1} , {θ}_{1} , {κ}_{2} , {θ}_{2} )\end{matrix} ] \\ = [ \begin{matrix}{f}_{a} ( {κ}_{1} , {θ}_{1} ) + {g}_{a} ( {κ}_{2} , {θ}_{2} ) + {i}_{a} ( {κ}_{1} , {θ}_{1} ) \times {j}_{a} ( {κ}_{2} , {θ}_{2} ) \\ {f}_{b} ( {κ}_{1} , {θ}_{1} ) + {g}_{b} ( {κ}_{2} , {θ}_{2} ) + {i}_{b} ( {κ}_{1} , {θ}_{1} ) \times {j}_{b} ( {κ}_{2} , {θ}_{2} ) \\ {f}_{c} ( {κ}_{1} , {θ}_{1} ) + {g}_{c} ( {κ}_{2} , {θ}_{2} ) + {i}_{c} ( {κ}_{1} , {θ}_{1} ) \times {j}_{c} ( {κ}_{2} , {θ}_{2} ) \\ {f}_{d} ( {κ}_{1} , {θ}_{1} ) + {g}_{d} ( {κ}_{2} , {θ}_{2} ) + {i}_{d} ( {κ}_{1} , {θ}_{1} ) \times {j}_{d} ( {κ}_{2} , {θ}_{2} )\end{matrix} ]\end{matrix} \qquad ( 7 )$$

Furthermore, each curved component S may be expressed as in the following equation (8). Compared with the equation (7), curvature information can be obtained with a small calculation load, and compared with the equation (5), more accurate curvature information can be obtained.

$$\begin{matrix}S = [ \begin{matrix}{S}_{a} ( {κ}_{1} , {θ}_{1} , {κ}_{2} , {θ}_{2} ) \\ {S}_{b} ( {κ}_{1} , {θ}_{1} , {κ}_{2} , {θ}_{2} ) \\ {S}_{c} ( {κ}_{1} , {θ}_{1} , {κ}_{2} , {θ}_{2} ) \\ {S}_{d} ( {κ}_{1} , {θ}_{1} , {κ}_{2} , {θ}_{2} )\end{matrix} ] \\ = [ \begin{matrix}{f}_{a} ( {κ}_{1} , {θ}_{1} ) + {g}_{a} ( {κ}_{2} , {θ}_{2} ) + {i}_{a} ( {κ}_{1} , {θ}_{2} ) + {j}_{a} ( {κ}_{2} , {θ}_{1} ) \\ {f}_{b} ( {κ}_{1} , {θ}_{1} ) + {g}_{b} ( {κ}_{2} , {θ}_{2} ) + {i}_{b} ( {κ}_{1} , {θ}_{2} ) + {j}_{b} ( {κ}_{2} , {θ}_{1} ) \\ {f}_{c} ( {κ}_{1} , {θ}_{1} ) + {g}_{c} ( {κ}_{2} , {θ}_{2} ) + {i}_{c} ( {κ}_{1} , {θ}_{2} ) + {j}_{c} ( {κ}_{2} , {θ}_{1} ) \\ {f}_{d} ( {κ}_{1} , {θ}_{1} ) + {g}_{d} ( {κ}_{2} , {θ}_{2} ) + {i}_{d} ( {κ}_{1} , {θ}_{2} ) + {j}_{d} ( {κ}_{2} , {θ}_{1} )\end{matrix} ]\end{matrix} \qquad ( 8 )$$

As described above, the curvature information derivation apparatus 10 according to the first embodiment is a curvature information derivation apparatus 10 that detects curvature information of a plurality of detection portions P1 and P2 in the longitudinal direction of the light guide 210, each of the plurality of detection portions includes at least one detection target 220 having a light absorber 214 that absorbs light of a specific wavelength, and the light absorber 214 included in the detection target 220 contains a color material selected so as to reduce the influence of an interaction caused by detection targets 220 at other detection portions existing in the light guide 210.

In this way, by selecting the color material contained in the light absorber 214 included in the respective detection targets 220, it becomes possible to obtain detected light quantity information that accurately indicates the bending state of each detection target 220 in the light detector 320, and determine curvature information (a direction of bending and a curvature) correctly, using the curvature information calculator 130, based on the detected light quantity information.

Here, the influence of the interaction can be reduced by making the particle size of the color material contained in the light absorber 214 included in each detection target 220 smaller than the specific wavelength.

In this case, the particle size of the color material is preferably ½ or less the specific wavelength.

Furthermore, the curvature information derivation apparatus 10 according to the first embodiment is a curvature information derivation apparatus 10 that detects curvature information of a plurality of detection portions P1 and P2 in the longitudinal direction of the light guide 210, each of the plurality of detection portions includes at least one detection target 220 having a light absorber 214 that absorbs light of a specific wavelength, and the light absorber 214 included in each detection target 220 is affected by an interaction caused by detection targets of other detection portions existing in the light guide 210. The curvature information derivation apparatus 10 includes a curvature information calculator 130 that derives curvature information of each of the detection portions by expressing a curved component S as an equation including all of curvature information pieces Sa, Sb, Sc, Sd, . . . in the detection portions P1 and P2 existing in the light guide 210.

Therefore, even if there is an influence of the interaction, the curvature information (a direction of bending and a curvature) can be determined correctly (an error is minimized).

### Second Embodiment

As described above, the magnitude of the influence of the interaction varies depending on the color material of the light absorber 214 to be used. The magnitude of the influence of the interaction can be evaluated by the light absorption properties of the respective light absorbers 214.

The more similar a light absorption property Ut in a transmission system and a light absorption property Ur in a reflection system are to each other, the smaller the influence of the interaction is. As a method for indicating whether the respective light absorption properties are similar, for example, there is a correlation coefficient J expressed by the following equation (9).

$$( 9 )$$

$$J = \frac{Covariance between Ut and Ur}{( Standard deviation of Ut ) ( Standard deviation of Ur )}$$

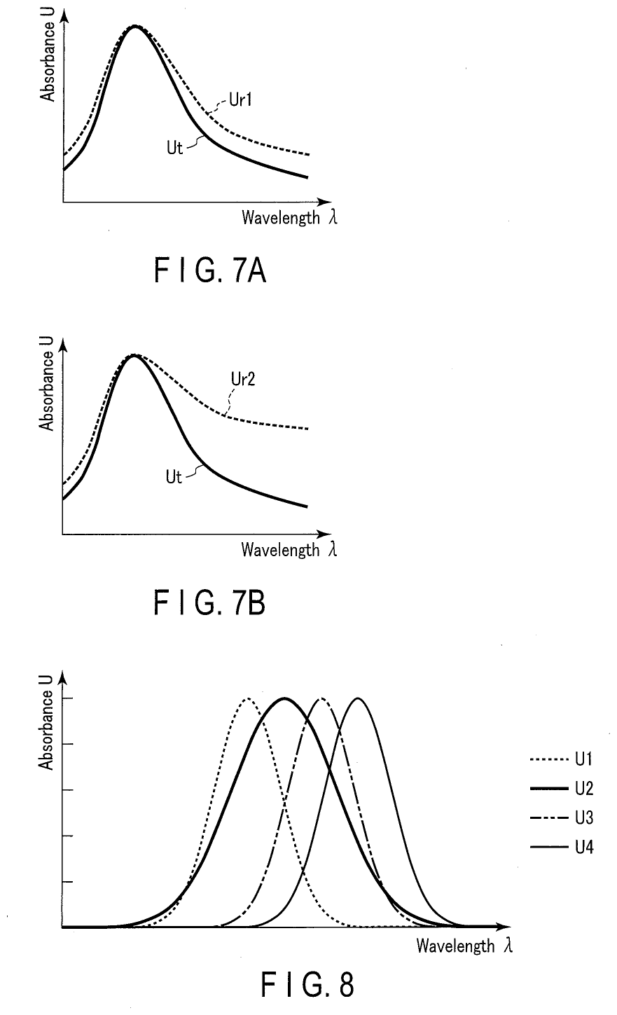

Comparing the light absorption property Ut in the transmission system and the light absorption property Ur (Ur1) in the reflection system as shown in FIG. 7A, with the light absorption property Ut in the transmission system and the light absorption property Ur (Ur2) in the reflection system as shown in FIG. 7B, the light absorption properties Ut and Ur1 shown in FIG. 7A have a stronger correlation. Therefore, for the light absorber 214, it is preferable to use a color material having a strong correlation, i.e., indicating a relationship of a high similarity, like the light absorption properties Ut and Ur1. In particular, when the correlation coefficient J is 0.6 or more, the influence of the interaction becomes negligible, which is preferable.

Thus, the influence of the interaction is reduced by selecting a color material of the light absorber 214 based on the similarity between the light absorption property Ut in a transmission system and the light absorption property Ur in a reflection system. As a result, the detected light quantity information acquired by the light detector 320 accurately indicates a bending state of the respective detection targets 220, and the curvature information calculator 130 can determine the curvature information correctly (the error of the determined curvature information can be minimized).

The light absorption property Ut of a color material in a transmission system and the light absorption property Ur of the color material in a reflection system can be acquired and selected by an experiment or a simulation.

As described above, the curvature information derivation device 10 according to the second embodiment can reduce the influence of the interaction by using, as a color material contained in the light absorber 214 included in each detection target 220, a color material having a high correlation between the light absorption property in a transmission system and the light absorption property in a light reflection system.

In this case, it is preferable that the correlation coefficient expressing the correlation be 0.6 or more.

### Third Embodiment

By using the color material as described in the first and second embodiments for the light absorber 214, the influence of the interaction can be reduced. However, there are cases where such a color material cannot be used. The third embodiment deals with such a case.

As described in the first embodiment, the configuration shown in FIG. 4 is affected by the bending state of another detection portion due to the interaction. Here, a case is considered where the light absorption property of a light absorber 214 disposed at one detection portion P1 is similar to the light absorption property of a light absorber 214 disposed at another detection portion P2.

When the similarity between the two light absorption properties is high, an interaction in the wavelength band which results in a property of absorption of each light absorber 214 occurs, so that the influence on the detected light quantity information acquired by the light detector 320 becomes large. Therefore, the light absorbers 214 having similar light absorption properties are arranged in the same detection portion (detection target group), not in different detection portions (detection target groups), thereby the influence of the interaction can be reduced.

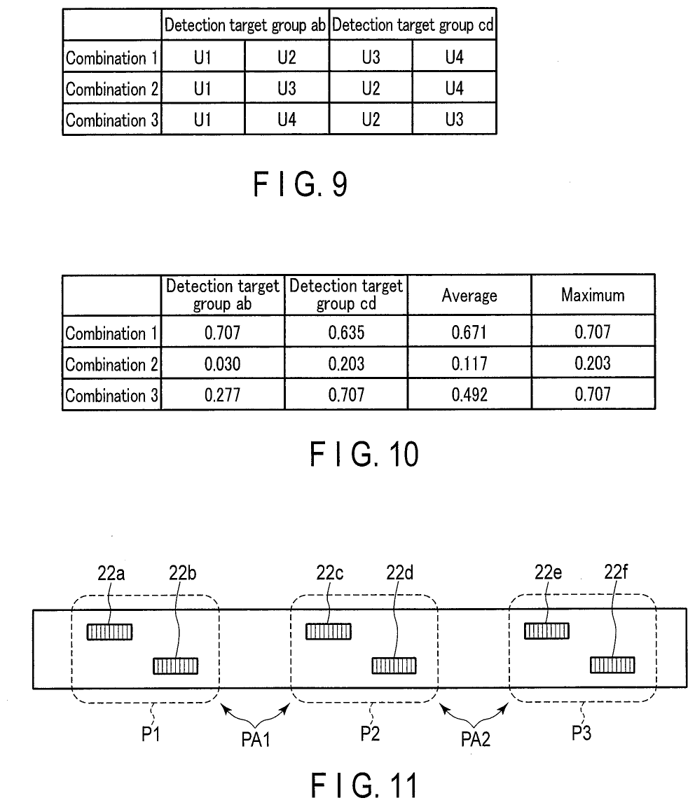

Here, a case is considered where four light absorbers U1 to U4 having light absorption properties as shown in FIG. 8 can be used as the four detection targets 22a, 22b, 22c, and 22d in the configuration of FIG. 4. As combinations of the arrangement of the four light absorbers U1 to U4, there exist three combinations 1 to 3 as shown in the table of FIG. 9 (in this case, they are not permutations).

There are various indexes for evaluating the similarity. It is possible to use, for example, a correlation coefficient J such as the equation (9) described in the second embodiment. When this correlation coefficient J is used, it suffices that the correlation coefficient J between the respective detection targets 220 arranged at each detection portion (detection target group) be increased.

When the correlation coefficients J for the combinations in the table shown in FIG. 9 are obtained, the table shown in FIG. 10 is obtained. From this table, a combination 1 is preferable because the average value and the maximum value of the correlation coefficient J are the largest. Therefore, in this example, light absorbers U1 and U2 are selected as the detection targets 22a and 22b of the detection target group ab at the detection portion P1, and light absorbers U3 and U4 are selected as the detection targets 22c and 22d of the detection target group cd at the detection portion P2.

In this way, by selecting and arranging the light absorbers 214 so that the correlation coefficient J between the respective detection targets 220 arranged at each detection portion (detection target group) is increased, the influence of the interaction of the light absorbers 214 arranged at one detection portion to the light absorbers 214 arranged at the other detection portion can be reduced, and an error in deriving curvature information can be reduced.

Furthermore, as shown in FIG. 11, the same applies to the case where there are three detection portions, P1, P2, and P3 (i.e., six detection targets 22a, 22b, 22c, 22d, 22e, and 22f) in the light guide 210. That is, it is only necessary to determine a combination so that the correlation coefficient J between two light absorbers 214 arranged at its detection portion (detection target group) is increased in each of the detection portion P1 (detection target group ab), the detection portion P2 (detection target group cd), and the detection portion P3 (detection target group ef).

The same applies to the case where there are four or more detection portions in the light guide 210.

In addition, when the distance between adjacent detection target groups (for example, the distance between a place where the detection target group ab is disposed and a place where the detection target group cd is disposed) is short, the probability that each detection target group is curved in the same direction becomes higher. For this reason, when the similarity of the light absorption properties of the light absorbers 214 arranged in the respective detection target groups is high, the light quantities in similar wavelength bands change simultaneously. Therefore, the change in the light quantity becomes large, and it becomes difficult to capture a slight change in the light quantity, causing an error in deriving curvature information.

Therefore, the light absorbers 214 are arranged so that the correlation coefficient J of the light absorption properties of the light absorbers 214 arranged in adjacent detection target groups is small. That is, in a pair PA1 of the detection portion P1 (detection target group ab) and the detection portion P2 (detection target group cd), a combination of the light absorbers is determined so that the correlation coefficient J between the detection target groups becomes small. Furthermore, in a pair PA2 of the detection portion P2 (detection target group cd) and the detection portion P3 (detection target group ef), a combination of the light absorbers is determined so that the correlation coefficient J between the detection target groups becomes small. By selecting the light absorber in this manner, it is possible to prevent the light quantities in similar wavelength bands from changing simultaneously, to easily measure a slight change in light quantity, and to reduce an error in deriving curvature information.

As described above, in the curvature information derivation apparatus 10 according to the third embodiment, it is assumed that each of the plurality of detection portions P1, P2, and P3 includes a plurality of detection targets 220 (22a, 22b; 22c, 22d; 22e, 22f), and a color material contained in the light absorber 214 included in each detection target 220 has a light absorption property that has a high correlation with the light absorption property of a color material included in the light absorbers included in another detection target of the same detection portion.

In this way, by combining the light absorbers 214 so that the similarity, e.g., the correlation, of the light absorbers 214 at the same detection portion is increased, the influence of the interaction between the light absorbers 214 arranged at one detection portion and the light absorbers 214 arranged at another detection portion can be reduced, and the error in deriving curvature information can be reduced.

In this case, the color material contained in the light absorber 214 included in each detection target 220 may be set to have a light absorption property that has a low correlation with the light absorption property of a color material contained in the light absorber included in a detection target of an adjacent detection portion.

In this way, by combining the light absorbers 214 so that the similarity, e.g., the correlation, of the light absorbers 214 at adjacent detection portions is lowered, it is possible to prevent light quantities in similar wavelength bands from changing simultaneously, to easily measure a change in the light quantity, and to reduce an error in deriving curvature information.

### Fourth Embodiment

Even when the influence of the interaction is reduced by using the color material as described in the first and second embodiments for the light absorbers 214, an error in deriving curvature information can be further reduced by taking into account the similarity of the light absorbers 214 in the same detection portion or the similarity of the light absorbers 214 in adjacent detection portions.

Similarly to the third embodiment, an example of the configuration as shown in FIG. 4 will be described.

Here, considering the case where four light absorbers U1 to U4 having the light absorption properties as shown in FIG. 8 can be used, there are three combinations 1 to 3 as shown in the table of FIG. 9. If the correlation coefficient J shown in the equation (9) is used as an index for evaluating the similarity as in the third embodiment, the combinations shown in FIG. 9 result in those shown in the table in FIG. 10.

When the influence of the interaction is small by applying, for example, the first or second embodiment, and if the similarity of the light absorption properties of a plurality of light absorbers 214 arranged in the same detection portion (detection target group) is high, the light quantities of similar wavelength bands change simultaneously. Therefore, the change in the light quantity becomes large, and it is difficult to capture a slight change in the light quantity, causing an error in deriving curvature information.

Therefore, if the similarity between the detection targets 220 arranged at the same detection portion (detection target group) is reduced, for example, if the correlation coefficient J is reduced, such a problem does not occur. From the table shown in FIG. 10, the combination 2 is preferable because the average value and the maximum value of the correlation coefficient J are the smallest. Therefore, in this example, light absorbers U1 and U3 are selected as the detection targets 22a and 22b of the detection target group ab at the detection portion P1, and light absorbers U2 and U4 are selected as the detection targets 22c and 22d of the detection target group cd at the detection portion P2.

As described above, by selecting and arranging the light absorbers 214 so that the correlation coefficient J between the respective detection targets 220 arranged in the same detection portion (detection target group) is small, a slight change in light quantity can be easily measured, and an error in deriving curvature information can be reduced.

In addition, as shown in FIG. 11, when there are three detection portions P1, P2, and P3 (i.e., six detection targets 22a, 22b, 22c, 22d, 22e, and 22f) in the light guide 210, what is necessary is also just to determine a combination so that the correlation coefficient J of the light absorbers 214 arranged at each of the detection target group ab, the detection target group cd, and the detection target group ef becomes small. The same applies to the case where there are four or more detection portions in the light guide 210.

Also in the fourth embodiment, similarly to the third embodiment, by arranging the light absorbers 214 so that the correlation coefficient J of the light absorption properties of the light absorbers 214 arranged in adjacent detection target groups becomes small, it is possible to prevent the light quantities in similar wavelength bands from changing, easily measure a slight change in light quantity, and reduce an error in deriving curvature information.

As described above, in the curvature information derivation apparatus 10 according to the fourth embodiment, each of the plurality of detection portions P1, P2, and P3 includes a plurality of detection targets 220 (22a, 22b; 22c, 22d; 22e, 22f), a color material contained in the light absorbers 214 included in the detection targets 220 is a color material having low correlation with the light absorption properties of a color material contained in a light absorber included in another detection target of the same detection portion.

In this way, by combining the light absorbers 214 so that the similarity, i.e., the correlation, of the light absorbers 214 in the same detection portion is lowered, it becomes easy to measure a slight change in light quantity, and an error in deriving curvature information can be minimized.

In addition, similarly to the third embodiment, in the curvature information derivation device 10 according to the fourth embodiment, a color material included in the light absorber 214 included in each detection target 220 may have a light absorption property having low correlation with the light absorption property of a color material contained in the light absorber included in a detection target of adjacent detection portion.

In this way, by combining the light absorbers 214 so that the similarity, i.e., the correlation, of the light absorbers 214 at adjacent detection portions is lowered, it is possible to prevent light quantities in similar wavelength bands from changing simultaneously, easily measure a change in the light quantity, and reduce an error in deriving curvature information.

### Fifth Embodiment

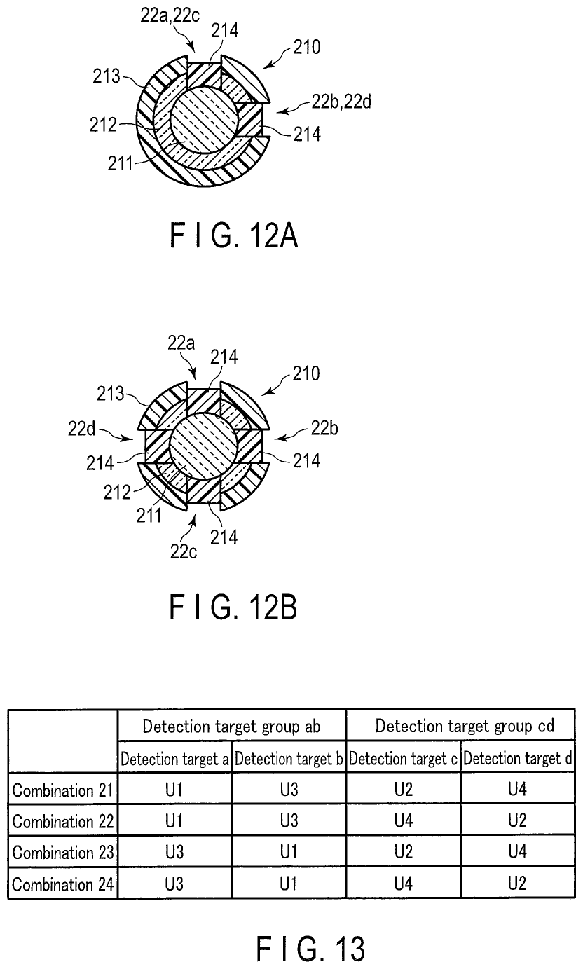

As shown in FIG. 4, when there are four detection targets 22a, 22b, 22c, and 22d in two detection portions (detection target groups) P1 and P2, it is possible to consider arranging these detection targets in the positional relationship as shown in FIG. 12A or FIG. 12B.

In FIG. 12A, in the cross section in the radial direction of the light guide 210, the two detection targets 22a and 22b of the detection portion P1 (detection target group ab) are arranged at positions orthogonal to each other. Here, the positions of the two detection targets 22a and 22b in the longitudinal direction of the light guide 210 may be the same, may partially overlap, or may be different. Similarly, the two detection targets 22c and 22d of the detection portion P2 (detection target group cd) are also arranged at positions orthogonal to each other in the cross section in the radial direction of the light guide 210. Furthermore, in the relationship between the detection target 22a of the detection portion P1 (detection target group ab) and the detection target 22c of the detection portion P2 (detection target group cd), these detection targets are arranged at the same position in the cross section in the radial direction of the light guides 210. Here, the positions of the two detection targets 22a and 22c in the longitudinal direction of the light guide 210 are naturally different. Similarly, the detection target 22b of the detection portion P1 (detection target group ab) and the detection target 22d of the detection portion P2 (detection target group cd) are also arranged at the same position in the cross section in the radial direction of the light guide 210.

In FIG. 12B, in the radial cross section of the light guide 210, four detection targets 22a, 22b, 22c, and 22d are arranged at two detection portions (detection target groups) P1 and P2 at positions orthogonal to each other.

With such an arrangement, when the distance between the detection target group ab and the detection target group cd is short, the probability that the detection targets 22a and 22c and the detection targets 22b and 22d are curved in the same direction is increased. For this reason, when the similarity of the light absorption properties of the light absorbers 214 arranged in the respective detection targets 220 is high, the change in the light quantity becomes large, and it is difficult to capture a slight change in the light quantity, causing an error in deriving curvature information.

When the influence of the interaction is small by applying the first or second embodiment as in the fourth embodiment, as described above, from the table shown in FIG. 10, the combination 2 is preferable because the average value and the maximum value of the correlation coefficient J are the smallest.

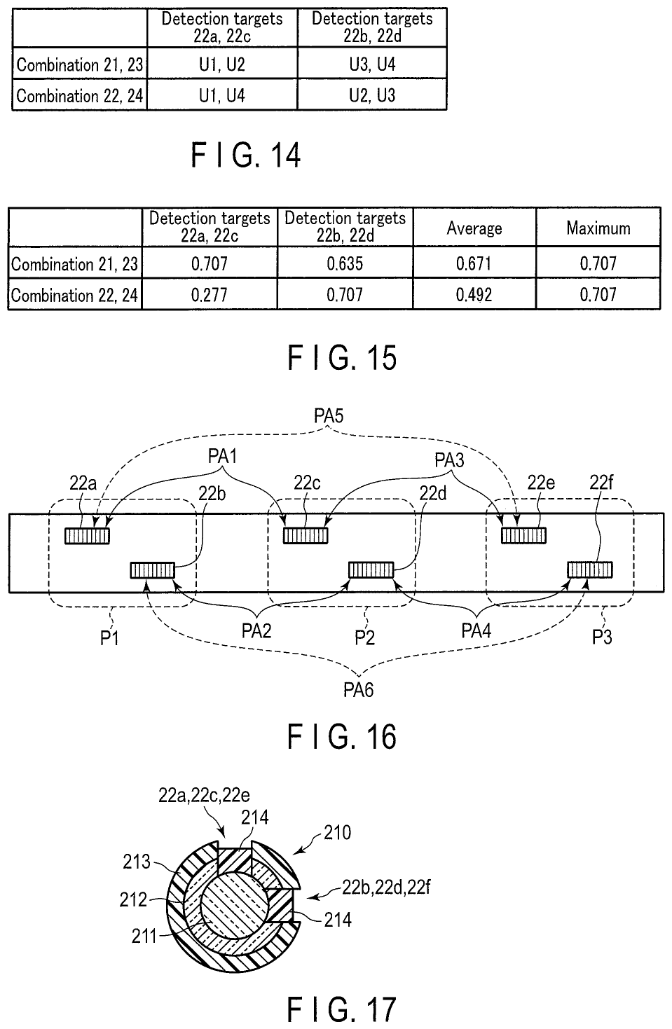

Considering the combination 2, there are four combinations of the arrangements of the four light absorbers U1 to U4 having the light absorption properties as shown in FIG. 8, and there are four combinations 21 to 24 as shown in the table of FIG. 13. Here, focusing on the combination of the detection targets 220 arranged in the same direction, the detection targets 22a and 22c and the detection targets 22b and 22d may be considered. Therefore, among these four combinations, it can be considered that the combination 21 and the combination 23 are the same, and the combination 22 and the combination 24 are the same. Therefore, as shown in the table of FIG. 14, they can be combined into two combinations.

When the correlation coefficient J is determined for these two combinations, the table shown in FIG. 15 is obtained. From the table shown in FIG. 15, it can be seen that the combination 22 and the combination 24 are preferable because the average value of the correlation coefficient J is small. Therefore, in this example, the light absorber U1 is selected as the detection target 22a of the detection target group ab at the detection portion P1, and the light absorber U4 is selected as the detection target 22c of the detection target group cd at the detection portion P2. The light absorber U3 is selected as the detection target 22b of the detection target group ab at the detection portion P1, and the light absorber U2 is selected as the detection target 22d of the detection target group cd at the detection portion P2. Alternatively, the light absorber U3 is selected as the detection target 22a of the detection target group ab at the detection portion P1, and the light absorber U4 is selected as the detection target 22c of the detection target group cd at the detection portion P2. The light absorber U1 is selected as the detection target 22b of the detection target group ab at the detection target P1, and the light absorber U2 is selected as the detection target 22d of the detection target group cd at the detection portion P2.

As described above, by selecting and arranging the light absorbers 214 so that the correlation coefficient J between the respective detection targets 220 arranged in the same detection portion (detection target group) is small, a slight change in light quantity can be easily measured, and an error in deriving curvature information can be reduced. Furthermore, by combining the light absorbers 214 so that the correlation coefficient J of the light absorbers 214 arranged in the same direction at adjacent detection portions is reduced, it is possible to prevent the light quantities in similar wavelength bands from changing simultaneously. Furthermore, it becomes easy to measure a slight change in the amount of light, and an error in deriving curvature information can be reduced.

Similarly, as shown in FIGS. 16 and 17, in the case where there are three detection portions P1, P2, and P3 (i.e., six detection targets 22a, 22b, 22c, 22d, 22e, and 22f) in the light guide 210, the combination is determined so that the correlation coefficient J of the light absorbers 214 arranged in each of the detection target group ab, the detection target group cd, and the detection target group ef is small.

That is, a combination is determined so that the correlation coefficient J is small in a pair PA1 of the detection target 22a of the detection portion P1 (detection target group ab) and the detection target 22c of the detection portion P2 (detection target group cd). A combination of the light absorbers is determined so that the correlation coefficient J becomes small in the pair PA2 of the detection target 22b of the detection portion P1 (detection target group ab) and the detection target 22d of the detection portion P2 (detection target group cd). Furthermore, a combination of the light absorbers is determined so that the correlation coefficient J becomes small in the pair PA3 of the detection target 22c of the detection portion P2 (detection target group cd) and the detection target 22e of the detection portion P3 (detection target group ef). A combination of the light absorbers is determined so that the correlation coefficient J becomes small in the pair PA4 of the detection target 22d of the detection portion P2 (detection target group cd) and the detection target 22f of the detection portion P3 (detection target group ef).

It should be noted that a pair PA5 of the detection target 22a of the detection portion P1 (detection target group ab) and the detection target 22e of the detection portion P3 (detection target group ef), and a pair PA6 of the detection target 22b of the detection portion P1 (detection target group ab) and the detection target 22f of the detected portion P3 (detection target group ef) are in the same direction, but the distance therebetween becomes large, so the priority in determining the combination may be low.

When the detection targets 22a, 22b, 22c, and 22d are arranged as shown in FIG. 12B, the light absorbers 214 having a high similarity to the opposite detection targets, that is, having a large correlation coefficient J are arranged, they operate to offset a change in the light quantities. For this reason, the change amount of light quantity becomes small, the detection range of the light detector 320 can be made smaller, and it becomes easy to capture a slight change in light quantity.

As described above, in the curvature information derivation apparatus 10 according to the fifth embodiment, a color material contained in the light absorber 214 included in the respective detection targets 220 has light absorption properties which are low in correlation with the light absorption properties of a color material contained in the light absorber included in other detection target arranged in the same direction at adjacent detection portion.

In this way, by combining the light absorbers 214 so that the similarity, e.g., the correlation, of the light absorbers 214 arranged in the same direction of adjacent detection portions is reduced, it becomes easy to measure a slight change in the light quantity, and an error in deriving curvature information can be reduced.

### Sixth Embodiment

The curvature information deriving method in the curvature information calculator 130 described in the first embodiment is also used in the second to fifth embodiments.

In the sixth embodiment, instead, another example of a method for deriving curvature information in the curvature information calculator 130 that can be used in the first to fifth embodiments will be described.

In this embodiment, a light quantity change rate at each wavelength or a logarithm V of the light quantity change rate, and a coefficient R for separating the V into curved components S of respective detection targets 220 are defined by the following equations (10) and (11). It should be noted that the curved component S indicating a bending state of each detection target 220 is defined as in equation (2) as described in the first embodiment.

$$\begin{matrix}V = [ \begin{matrix}{V}_{λ1} \\ {V}_{λ2} \\ ⋮ \\ {V}_{λ n} \\ {V}_{λ1}^{2} \\ {V}_{λ2}^{2} \\ ⋮ \\ {V}_{λ n}^{2} \\ {V}_{λ 1} \times {V}_{λ 2} \\ {V}_{λ 1} \times {V}_{λ 3} \\ ⋮ \\ {V}_{λ ( n - 1 )} \times {V}_{λ n}\end{matrix} ] & ( 10 ) \\ R = [ \begin{matrix}{R}_{a λ1} & {R}_{a λ 2} & … & {R}_{a λ ( n - 1 ) n} \\ {R}_{b λ1} & ⋱ & & \\ ⋮ & & & \\ {R}_{X λ 1} & & & {R}_{X λ ( n - 1 ) n}\end{matrix} ] & ( 11 )\end{matrix}$$

As shown in equation (10), an element of V is expressed not only by a change rate V_{λn} at each wavelength, but also by a square of the change rate V_{λn} and a product of change rates at respective wavelengths. A constant (≠0) may be added as an element, although this is not described. In addition, a combination of higher order change rates may be further added as an element.

Similar to the first embodiment, the curvature information calculator 130 separates the detected light quantity information acquired by the light detector 320 into a curved component S of each of the detection targets 220 by equation (4).

Hereinafter, as a specific example, a configuration having four detection targets 22a, 22b, 22c, and 22d as shown in FIG. 4 will be described. Two detection targets 220 (two detection portions P1 and P2) are arranged on the light guide 210. A detection target group ab at the detection portion P1 is curved with a curvature θ₁ and a direction of bending κ₁, and a detection target group cd at a detection portion P2 is curved with a curvature θ₂ and a direction of bending κ₂.

In this case, each curved component S can be expressed by a function of the curvature information pertaining to it, as in the following equation (12).

$$S = [ \begin{matrix}{S}_{a} ( {κ}_{1} , {θ}_{1} ) \\ {S}_{b} ( {κ}_{1} , {θ}_{1} ) \\ {S}_{c} ( {κ}_{2} , {θ}_{2} ) \\ {S}_{d} ( {κ}_{2} , {θ}_{2} )\end{matrix} ] = [ \begin{matrix}{f}_{a} ( {κ}_{1} , {θ}_{1} ) \\ {f}_{b} ( {κ}_{1} , {θ}_{1} ) \\ {f}_{c} ( {κ}_{2} , {θ}_{2} ) \\ {f}_{d} ( {κ}_{2} , {θ}_{2} )\end{matrix} ] \qquad ( 12 )$$

By expressing the change rate of each wavelength as in equation (10), curvature information can be derived even if there is an interaction. For this reason, curvature information can be calculated correctly (an error is minimized).

Each curved component S may be expressed by a function of the curvature information pertaining to it and the other curvature information as shown in Equation (5). More accurate curvature information may be obtained by expressing the curved component as an equation including the curvature information of all of the detection portions as in equation (5).

As described above, the curvature information derivation apparatus 10 according to the sixth embodiment is a curvature information derivation apparatus 10 that detects curvature information of a plurality of detection portions P1 and P2 in the longitudinal direction of the light guide 210. Each of the plurality of detection portions includes at least one detection target 220 having a light absorber 214 that absorbs light of a specific wavelength, and the light absorber 214 is affected by an influence of an interaction caused by a detection target at another detection portion existing in the light guide 210. The curvature information derivation apparatus 10 includes a curvature information calculator 130 that derives curvature information at the respective detection portions by configuring a separation coefficient for separating detected curvature information into a curved component S as respective change rates V_{λn}, V_{λn}² of each of the specific wavelengths in the light absorbers included in the detection targets of all of the detection portions existing in the light guide 210 and a combination of a change rage of each wavelength, for example, a product of change rates at respective wavelengths.

Therefore, even if there is an influence of the interaction, accurate curvature information (a direction of bending and a curvature) can be determined (an error is minimized).

Of course, also in the curvature information derivation apparatus 10 according to the sixth embodiment, the curvature information calculator 130 can derive curvature information at respective detection portions by expressing a curvature component S by an equation including curvature information Sa, Sb, Sc, Sd, . . . of all of detection portions P1, P2, . . . existing in the light guide 210.

Additional advantages and modifications will readily occur to those skilled in the art. Therefore, the invention in its broader aspects is not limited to the specific details, representative devices, and illustrated examples shown and described herein. Accordingly, various modifications may be made without departing from the spirit or scope of the general inventive concept as defined by the appended claims and their equivalents.

What is claimed is:

1. A fiber sensor comprising:
a light guide provided with a plurality of detection portions in a longitudinal direction of the light guide; and
a plurality of detection targets each having a light absorber that absorbs light of a specific wavelength, each of the plurality of detection portions including at least one detection target, wherein
each of the light absorbers contains a color material that reduces an influence of an interaction caused by each detection target at another detection portion existing in the light guide.

2. The fiber sensor according to claim 1, wherein each of the plurality of the detection portions includes a plurality of detection targets, and
the color material contained in the light absorber included in each of the detection targets has a light absorption property which is high in correlation with a light absorption property of a color material contained in a light absorber included in another detection target of the same detection portion.

3. The fiber sensor according to claim 1, wherein the color material contained in the light absorber included in the detection target has high correlation between a light absorption property in a transmission system and a light absorption property in a reflection system.

4. The fiber sensor according to claim 3, having a correlation coefficient indicating the correlation of 0.6 or more.

5. The fiber sensor according to claim 1, wherein the color material contained in the light absorber included in the detection target has a particle size smaller than the specific wavelength.

6. The fiber sensor according to claim 5, wherein the particle size of the color material is ½ or less the specific wavelength.

7. The fiber sensor according to claim 1, wherein each of the plurality of detection portions includes a plurality of detection targets, and
the color material contained in the light absorber included in each of the detection targets has a light absorption property which is low in correlation with a light absorption property of a color material contained in a light absorber included in another detection target in the same detection portion.

8. The fiber sensor according to claim 7, wherein the color material contained in the light absorber included in the detection target has a light absorption property which is low in correlation with a light absorption property of a color material contained in light absorber included in each of the detection targets in an adjacent detection portion.

9. The fiber sensor according to claim 7, wherein the color material contained in the light absorber included in the detection target has a light absorption property which is low in correlation with a light absorption property of a color material contained in light absorbers included in the other detection targets arranged in the same direction of an adjacent detection portion.

10. An endoscope system comprising:
a curvature information derivation apparatus that comprises the fiber sensor according to claim 1, and derives curvature information including a direction of bending and a size of bending by detecting light transmitted by the light guide of the fiber sensor; and
an endoscope including an insertion portion in which the light guide is incorporated.

11. A curvature information derivation apparatus comprising:
a fiber sensor including a light guide provided with a plurality of detection portions in a longitudinal direction of the light guide; and
a curvature information calculator configured to derive curvature information of each of the plurality of detection portions, wherein
each of the plurality of detection portions including at least one detection target having a light absorber that absorbs light of a specific wavelength,
each of the light absorbers is affected by an interaction caused by a detection target at another detection portion, and
the curvature information calculator derives the curvature information of each of the plurality of detection portions by expressing a curved component with an equation including curvature information of all of detection portions existing in the light guide.

12. An endoscope system comprising:
the curvature information derivation apparatus according to claim 11, and
an endoscope including an insertion portion in which the light guide is incorporated.

13. A method for manufacturing a fiber sensor including a light guide which has a core, a clad surrounding the core and a jacket surrounding the clad, and is provided with a plurality of detection portions in a longitudinal direction of the light guide, the method comprising:
removing the jacket and the clad corresponding to each of a plurality of detection targets to expose the core, each of the plurality of detection portions including at least one detection target; and
providing a light absorber on the core corresponding to each of the detection targets and exposed, wherein
each of the light absorbers contains a color material that reduces an influence of an interaction caused by each detection target at another detection portion existing in the light guide.

14. The method according to claim 13, wherein each of the plurality of the detection portions includes a plurality of detection targets, and
the color material contained in the light absorber included in each of the detection targets has a light absorption property which is high in correlation with a light absorption property of a color material contained in a light absorber included in another detection target of the same detection portion.

15. The method according to claim 13, wherein the color material contained in the light absorber included in the detection target has high correlation between a light absorption property in a transmission system and a light absorption property in a reflection system.

16. The method according to claim 15, having a correlation coefficient indicating the correlation of 0.6 or more.

17. The method according to claim 13, wherein the color material contained in the light absorber included in the detection target has a particle size smaller than the specific wavelength.

18. The method according to claim 17, wherein the particle size of the color material is ½ or less the specific wavelength.

19. The method according to claim 13, wherein each of the plurality of detection portions includes a plurality of detection targets, and
the color material contained in the light absorber included in each of the detection target has a light absorption property which is low in correlation with a light absorption property of a color material contained in a light absorber included in another detection target in the same detection portion.
